SEQUENCE LISTING (3 sequences)

SEQ ID NO: 1 - protein, 80 aa, Homo sapiens
SWKPRSQQPDAPLGTGANRDLELPWLEQQGPASHHRRQLGPQGPPHLVADPSKKQGPWLEEEEEAYGWMDFGRRSAEDEN

SEQ ID NO: 2 - protein, 14 aa, artificial
  Amino acids 1-14, N-terminal extremity of human progastrin
SWKPRSQQPDAPLG

SEQ ID NO: 3 - protein, 26 aa, artificial
  Amino acids 55-80, C-terminal extremity of human progastrin
QGPWLEEEEEAYGWMDFGRRSAEDEN

(19) United States
(12) Patent Application Publication
(10) Pub. No.: US 2020/0390913 A1
(43) Pub. Date: Dec. 17, 2020

(54) RADIOLABELED PROGASTRIN IN CANCER DIAGNOSIS

(71) Applicant: ECS-Biotracker SÀRL, Prilly (CH)

(72) Inventor: Jean-François Floch, Garlan (FR)

(73) Assignee: ECS-Biotracker SÀRL, Prilly (CH)

(21) Appl. No.: 16/770,180

(22) PCT Filed: Dec. 10, 2018
(86) PCT No.: PCT/EP2018/084172

§ 371 Date: Jun. 5, 2020

Related U.S. Application Data
(60) Provisional application No. 62/596,196, filed on Dec. 8, 2017.

(51) Int. Cl. A61K 51/08; C07K 14/595; C07K 19/00; C07K 1/13

(52) U.S. Cl. CPC A61K 51/088; C07K 1/13; C07K 19/00; C07K 14/595

(57) ABSTRACT

The present invention provides a radiotracer comprising a progastrin moiety, a chelating moiety and a radioisotope. Uses of said biomarker for imaging and detecting cancers in a subject are also provided. In one embodiment, the radiotracer is ⁶⁸Ga-NODAGA-Progastrin.

### INTRODUCTION

Cancer is a multi-faceted disease in which a group of cells display uncontrolled growth, invasion that intrudes upon and destroys adjacent tissues, and sometimes metastasis, or spreading to other locations in the body via lymph or blood. These three malignant properties of cancers differentiate them from benign tumours, which do not invade or metastasize.

There are a number of methods currently used to treat each type of cancer, including surgery, radiotherapy, chemotherapy, targeted therapy and immunotherapy. Successful cancer therapy is directed to the primary tumour and to any metastases, whether clinically apparent or microscopic.

It is crucial for the patient to identify as early as possible the type of cancer to be treated. Cancer that is diagnosed at an early stage, is more likely to be treated successfully. If cancer spreads, effective treatment becomes more difficult, and generally chances of surviving are much lower. So, it is essential to know when to use immediately a heavy and aggressive treatment protocol in order to prevent extension of an aggressive cancer.

Currently, treatment selection for solid tumours is based on tumour staging, which is usually performed using the Tumour/Node/Metastasis (TNM) test from the American Joint Committee on Cancer (AJCC). It is commonly acknowledged that, while this test and staging system provides some valuable information concerning the stage at which solid cancer has been diagnosed in the patient, it is imprecise and insufficient. In particular, it is limited to solid tumours.

Most importantly, the TNM test fails to identify the earliest stages of tumour progression. These early stages offer the most promising window for therapy. Detection of a cancer at the very beginning of its development allows targeted, efficient therapy, with reduced side-effects. It is thus important to identify patients at the earliest possible stage as a part of a screening of the whole population. Cancer can thus be identified in a community early, enabling earlier intervention and management to reduce mortality and suffering from said disease.

A diagnosis test based on the detection of progastrin has recently been developed by the applicant. Selected antibodies were used to set up an ELISA assay to detect progastrin in the blood of patients with various types of cancers and at various stages. This test, commercialized under the name CancerRead, is particularly efficient for detecting various types of cancer, including early stages (WO 2017/114973). Notably, the CancerRead test displays high sensitivity and specificity for early stage tumours.

However, even though the level of progastrin in blood is a reliable biomarker for early cancer screening, it gives no information regarding the origin of the cancer.

There is thus a real need for reagents allowing in vivo identification of a cancer, so that an appropriate therapy can be provided at the earliest possible stage.

### DESCRIPTION

The present invention relates to a derivative of progastrin for imaging a cancer in patient.

In a first aspect, the present invention relates to a compound, or a pharmaceutically acceptable salt thereof, said compound comprising:
a progastrin moiety, and
a chelating moiety,

wherein said chelating moiety is optionally associated with a radioisotope.

In a preferred embodiment, the progastrin moiety and the chelating moiety are covalently linked. According to this embodiment, the present compound is a conjugate.

The compounds of the invention are particularly useful because they are capable of binding to cancer cells in vivo, thus enabling the imaging of said cancer. This is particularly advantageous for identifying the localisation of a cancer. Notably, the radiolabelled progastrin are used for flow visualisation through different technologies, such as Single Photon Emission Computed Tomography (SPECT) and Positron Emission Tomography (PET).

By “progastrin”, it is herein referred to the mammalian progastrin peptide. Progastrin is formed by cleavage of the first 21 amino acids (the signal peptide) from preprogastrin, a 101 amino acids peptide (Amino acid sequence reference: AAB19304.1) which is the primary translation product of the gastrin gene. The 80 amino acid chain of progastrin is further processed by cleavage and modifying enzymes to several biologically active gastrin hormone forms: gastrin 34 (G34) and glycine-extended gastrin 34 (G34-Gly), comprising amino acids 38-71 of progastrin, gastrin 17 (G17) and glycine-extended gastrin 17 (G17-Gly), comprising amino acids 55 to 71 of progastrin.

In a preferred embodiment, the progastrin derivative is a derivative of human progastrin. More preferably, the expression “human progastrin” refers to the human progastrin of sequence SEQ ID No. 1. Human progastrin comprises notably a N-terminus and a C-terminus domain, both of which are not present in the biologically active gastrin hormone forms mentioned above. Preferably, the sequence of said N-terminus domain is represented by SEQ ID NO. 2. In another preferred embodiment, the sequence of said C-terminus domain is represented by SEQ ID NO. 3.

Gastrin cells naturally produce progastrin, which is maturated into gastrin. During digestion, 95% of progastrin is released as gastrin from the cell. A very small amount of progastrin is released as progastrin. Hence, except during digestion, healthy people have no progastrin in their blood.

On the other hand, in pathological conditions, progastrin becomes an early marker. In tumour cells, progastrin is not maturated into gastrin and is consequently released from the tumoural cell. Progastrin can promote tumourigenesis (e.g. gastric [Burkitt et al., World J Gastroenterol. 15(1): 1-16, 2009, WO 2017/114975], colon [Watson et al., J Cancer. 87(5): 567-573, 2002], pancreatic [Harris et al., Cancer Res. 64(16): 5624-5631, 2004, WO 2011/083091], ovarian [WO 2017/114972], prostate [WO 2018/178352], oesophageal [WO 2017/114976], and lung cancers [WO 2018/178354]) in an autocrine, paracrine or endocrine manner (Dimaline a Varro, J Physiol 592(Pt. 14): 2951-2958, 2014), which has also warranted progastrin as a preferred anti-tumour target in cancers expressing these stimulatory factors (see e.g., WO 2011/045080, WO 2011/083088, WO 2011/116954, WO 2012/013609, WO 2011/083090, WO 2011/083091, WO 2017/114975, WO 2017/114976, WO 2017/114972, WO 2018/178364). This process is independent of digestion.

A “chelating moiety” or “chelating agent” or “chelator” as used herein refers to a compound which is capable of chelating any of these radioisotopes. The chelating moiety sequesters the corresponding free radioisotopes from aqueous solutions, thus enabling applying said isotopes to specific biological applications. Preferably, said chelating moiety is a bifunctional chelator. A “bifunctional chelator or “bifunctional chelating agent” as used herein refers to a compound possessing a metal binding moiety function and a chemically reactive functional group.

Numerous bifunctional chelators are known in the art. A great number of them are indeed available commercially and have been routinely used as PET imaging agents. The structure and physical properties vary between bifunctional chelators. The skilled person will select the most appropriate bifunctional chelator for using with the progastrin moiety, taking notably into account the radioisotope which is used (see e.g., Cutler et al., Chem Rev. 113(2): 858-883, 2013; Price & Orvig, Chem. Soc. Rev. 43(1): 260-290, 2013; Tornesello et al., Molecules 22: E1282, 2017; Brandt et al., J Nucl Med 59(10): 1500-1506, 2018; Morais & Ma, Drug Discovery Today: Technologies, 2018, DOI: 10.1016/j.ddtec.2018.10.002).

Example of bifunctional chelating agents are represented in Table 1.

| Chelator | Structure |
|---|---|
| NODAGA | 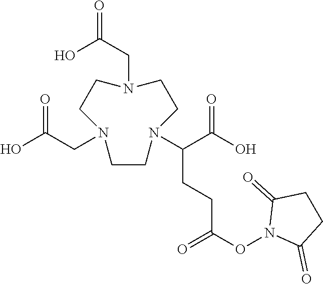 [SMILES: O=C(O)CN1CCN(CC(=O)O)CCN(C(CCC(=O)ON2C(=O)CCC2=O)C(=O)O)CC1] |
| DOTA | 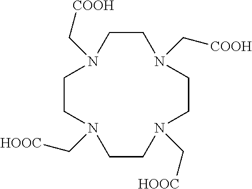 [SMILES: O=C(O)CN1CCN(CC(=O)O)CCN(CC(=O)O)CCN(CC(=O)O)CC1] |
| DOTA-NHS | 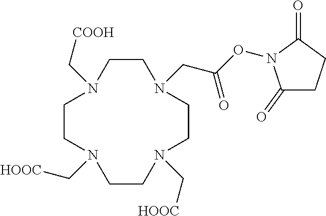 [SMILES: O=C(O)CN1CCN(CC(=O)O)CCN(CC(=O)ON2C(=O)CCC2=O)CCN(CC(=O)O)CC1] |
| p-SCN-Bn-NOTA | 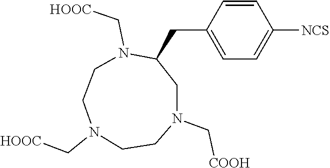 |
| p-SCN-Bn-PCTA | 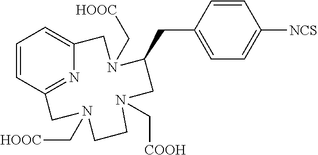 |
| p-SCN-Bn-oxo-DO3A | 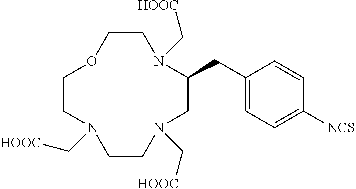 |
| desferrioxamine-p-SCN | 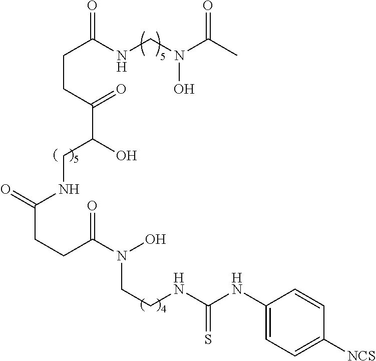 |
| Diethylenetriamine Pentaacetic Acid (DTPA) | 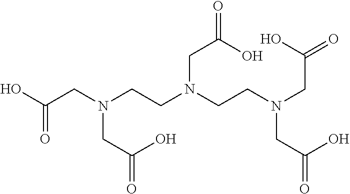 [SMILES: O=C(O)CN(CCN(CC(=O)O)CC(=O)O)CCN(CC(=O)O)CC(=O)O] |
| 1,4,8,11- Tetraazacyclotetradecane- 1,4,8,11-tetraacetic acid (TETA) | 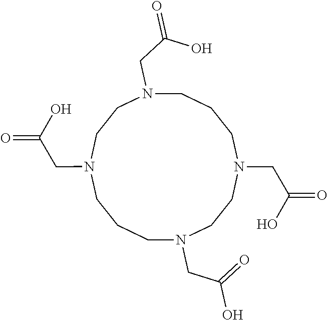 [SMILES: O=C(O)CN1CCCN(CC(=O)O)CCN(CC(=O)O)CCCN(CC(=O)O)CC1] |

The bifunctional chelator is thus preferably selected in the list of NODAGA, NOTA, DOTA, DOTA-NHS, p-SCN-Bn-NOTA, p-SCN-Bn-PCTA, p-SCN-Bn-oxo-DO3A, desferrioxamine-p-SCN, DTPA, and TETA.

DOTA, NOTA and NOGADA are commonly used bifunctional chelators, notably for ⁶⁸Ga labelling. Thus, fast and quantitative ⁶⁸Ga-radiolabeling of biomolecules can be achieved by the employment of well-known chelators such as DOTA, NOTA, and NOGADA.

In particular, it has been shown that the chelating agent, DOTA (1,4,7,10-tetraazacyclododecane-1,4,7,10-tetraacetic acid), (or modified derivatives thereof), is an excellent ligand for binding of gallium; and DOTA-peptides can be rapidly and efficiently labelled with ⁶⁸Ga at high specific activities (Velikyan, Molecules, 20: 12913-12943, 2015). Likewise, diethylenetriamine pentaacetic acid (DTPA) and its derivative have been widely used. For example, the 1B4M-DTPA, also known as MX-DTPA or tiuxetan, has been developed as the chelating agent component of Zevalin for radiolabeling with either ¹¹¹In or ⁹⁰Y (Brechbiel, Q J Nucl Med Mol Imaging. 52(2): 166-173, 2008).

NOTA (4,7-triazacyclononane-1,4,7-triacetic acid) is generally considered to be the “gold standard” for Ga3+ chelation, possessing favorable radiolabeling conditions (RT, 30-60 minutes) and excellent in vivo stability. Indeed, NOTA and derivatives are well-known to form very stable complexes with ⁶⁸Ga and with ⁶⁴Cu.

Derivatives of NOTA, especially NODAGA (1,4,7-triazacyclononane-1-glutaric acid-4,7-diacetic acid) have proven to be more suitable for chelating the ⁶⁸Ga ion than those of DOTA. NODAGA is particularly useful for ⁶⁸Ga- and ⁶⁴Cu-labeling due to high hydrophilicity and in vivo stability of its ⁶⁸Ga and ⁶⁴Cu chelates. Clinical studies have demonstrated that radiotracers containing [⁶⁸Ga]NODAGA are well tolerated without drug-related adverse effects in patients (see e.g., Haubner et al., Eur J Nucl Med Mol Imaging 43:2005-2013, 2016; Kumar et al., J Nucl Med 57(suppl. 2): 1171, 2016; Ben Azzouna et al., Endocrine Abstracts 47: OC4, 2016). Indeed, [⁶⁸Ga]NODAGA appear to be particularly suited for tumour imaging in vivo (see e.g., Oxboel et al., Nucl Med Biol. 41(3):259-267, 2014; Kumar et al., J Nucl Med 57(suppl. 2): 675, 2016; Kumar et al., J Nucl Med 57(suppl. 2): 1171, 2016; Kumar et al., J Nucl Med 57(suppl. 2): 1298, 2016; Tornesello et al., Molecules 22: E1282, 2017). NODAGA is commercially available from different suppliers as NODAGA-NHS esters, allowing simple bioconjugation to an amine of the progastrin moiety.

Preferably, the chelating agent is selected between DOPA, NOTA, and NODAGA. Most preferably, the chelating agent is NODAGA.

A “radioisotope” as used herein is a version of a chemical element that has an unstable nucleus and emits radiation during its decay to a stable form. Radioisotopes have important uses in medical diagnosis, treatment, and research. The radioisotope of the present compounds is preferably selected in the list consisting of ⁶⁸Ga, ⁶⁴Cu, ⁸⁹Zr, ^{186/188}Re, ⁹⁰Y, ¹⁷⁷Lu, ¹⁵³Sm, ²¹³Bi, ²²⁵A, ¹¹¹In, ^{99m}Tc, ¹²³I, or ²²³Ra. These radioisotopes are particularly advantageous because of their long half-life and small size, which makes them particularly suitable for PET/SPECT imaging. More preferably, the radioisotope is ⁶⁸Ga or ⁶⁴Cu. Even more preferably, said radioisotope is ⁶⁸Ga.

The advantages of ⁶⁸Ga over other PET-based radionuclides include notably its availability from an in-house generator independent of an onsite cyclotron (Shukla & Mittal, J Postgrad Med Edu Res 47(1): 74-76, 2013). It can thus be cost effectively and continuously produced by a commercially available ⁶⁸Ge/⁶⁸Ga generator, alleviating the need for proximity of PET centres to the cyclotrons needed for the production of, for example, ¹⁸F. The disintegration mode of the radionuclide results in high quality positron emission tomography (PET) images and allows accurate quantification. In addition, the short physical half-life of ⁶⁸Ga (t_{1/2}=68 min) enables improved dosimetry and repeat imaging, making these agents ideal for clinical use. Notably, this half-life facilitates imaging soon after administration with reduced exposure to the patient. Small compounds, biological macromolecules as well as nano- and micro-particles have been successfully labelled with ⁶⁸Ga, and the resulting agents demonstrated promising imaging capability pre-clinically and clinically (see e.g., Beylergil et al., Nucl Med Commun. 34(12): 1157-1165, 2013).

Other embodiments of the disclosure include pharmaceutically acceptable salts of the compounds described in any of the previous embodiments. As used herein, “pharmaceutically acceptable salts” refer to derivatives of the disclosed compounds wherein the parent compound is modified by making non-toxic acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, malefic, hydroxymaleic, phenylacetic, glutamic, benzoic, salicylic, mesylic, sulfanilic, 2-acetoxybenzoic, fumaric, toluenesulfonic, methanesulfonic, ethane disulfonic, oxalic, isethionic, HOOC—(CH2)ₙ—COOH where n is 0-4, and the like. The pharmaceutically acceptable salts of the present disclosure can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are used, where practicable. Lists of additional suitable salts may be found, e.g., in Remington's Pharmaceutical Sciences, 17th ed., Mack Publishing Company, Easton, Pa., p. 1418 (1985).

In another aspect, the present invention provides a method of preparing the compound of the invention. Said method comprises the steps of:
a) conjugating an amine-reactive chelating moiety to the progastrin moiety; and
b) recovering the conjugate of progastrin and chelator.

Amine-reactive chelate structures for the radioisotope described herein are commercially available, such as e.g., DOTA-NHS, NOTA-NHS, and NODAGA-NHS esters. Preferably, said amine-reactive chelating moiety is an NODAGA-NHS ester. It is well known to the person of skill in the art that NHS esters (N-hydroxysuccinimide esters) will react with primary amines at the N-terminus and in the side-chain of lysine (Lys, K) amino acid residues of the progastrin residues, and thus needs not to be detailed here.

Preferably, the method of preparing the compound of the invention further comprises a step of:
c) incubating the conjugate of progastrin and chelator with the complementary radioisotope;

thus generating the compound of the invention.

In another aspect, the present invention provides a method of imaging one or more cells, organs or tissues by exposing the cell to or administering to an organism an effective amount of the compound, where the compound includes a metal isotope suitable for imaging. Imaging can be performed by any suitable technique known to the person skilled in the art, notably PET or SPECT.

SPECT and PET are functional imaging techniques used to localize metabolic processes. A radionuclide produced from either a cyclotron or a generator is attached to a biologically active molecule forming a PET radiotracer. Isotopes that are currently used in SPECT/PET imaging studies are attractive and potentially better alternatives to ¹⁸F. ⁶⁸Ga, ⁶⁴Cu, ⁸⁹Zr, ^{186/188}Re, ⁹⁰Y, ¹⁷⁷Lu, ¹⁵³Sm, ²¹³Bi, ²²⁵Ac, or ²²³Ra are available isotopes that are being assessed for PET imaging due to their light metal properties and the ability to bind to chelating agents.

Positron emission tomography (PET) is a nuclear medicine, functional imaging technique that produces a three-dimensional image of functional processes in the body. PET is used to localize metabolic processes. A positron-emitting radionuclide produced from either a cyclotron or a generator is attached to a biologically active molecule forming a PET radiotracer, such as e.g., the compounds described herein. The PET radiotracer is then introduced into the patient by injection, ingestion, or inhalation. The system detects pairs of gamma rays emitted indirectly by a radionuclide (tracer), which is introduced into the body on the radiotracer. Three-dimensional images of tracer concentration within the body are then constructed by computer analysis. In modern PET-CT scanners, three-dimensional imaging is often accomplished with the aid of a CT X-ray scan performed on the patient during the same session, in the same machine. Once the PET radiotracer is administered, the patient is positioned so that detectors can register incident gamma rays, 2 511 keV photons traveling in opposite directions, produced as the radionuclide decays resulting in an annihilation event from a positron combining with an electron after traversing a short distance. The detector's electronics are synced in such a way that the 2 photons emitted are detected on opposite sides and are called coincident and therefore must have originated from the same annihilation event. These coincident projections are assigned to a line of response and are then reconstructed using standard tomographic techniques to identify the location of the annihilation event. By using modern “time of flight” information in PET image reconstruction with very fast scintillators, the origin of the annihilation event along the line of response is detected with improved accuracy.

Radionuclides used in PET scanning are typically isotopes with short half-lives such as ¹¹C (˜20 min), ¹³N (˜10 min), ¹⁵O (˜2 min), ¹⁸F (˜110 min), or ⁸²Rb (˜1.27 min). The radioisotopes described above, i.e., the list consisting of ⁶⁸Ga, ⁶⁴Cu, ⁸⁹Zr, ^{186/188}Re, ⁹⁰Y, ¹⁷⁷Lu, ¹⁵³Sm, ²¹³Bi, ²²⁵Ac, or ²²³Ra, are also commonly used in PET. In this regard, as noted above, ⁶⁸Ga is particularly advantageous because of its half-life of 68 minutes. These radionuclides are incorporated either into compounds normally used by the body such as glucose (or glucose analogues), water, or ammonia, or into molecules that bind to receptors or other sites, including progastrin. Such labelled compounds are known as radiotracers. PET technology can be used to trace the biologic pathway of any compound in living humans (and many other species as well), provided it can be radiolabelled with a PET isotope. In particular, as described below, PET technology can be used to detect a cancer in a living human by imaging of radiolabelled probe which binds specifically to cancerous cells, such as the compound described herein.

Due to the short half-lives of most positron-emitting radioisotopes, the radiotracers have traditionally been produced using a cyclotron in close proximity to the PET imaging facility. The half-life of fluorine-18 is long enough that radiotracers labelled with fluorine-18 can be manufactured commercially at offsite locations and shipped to imaging centres. On the other hand, ⁶⁸Ga can be produced in a generator, thus disposing with the need of a cyclotron (Velikyan, Molecules 20: 12913-12943, 2015). In addition, the half-life of gallium-68 is close to the one of ¹⁸F, making this radionuclide particularly useful for PET imaging.

Single-photon emission computed tomography (SPECT) is nuclear medicine imaging technique similar to PET. It also uses a radioactively labelled tracer and is based on the detection of gamma rays. In contrast to PET, the radioactive label used in SPECT emits a gamma radiation that is measured directly.

Embodiments of the invention include the present compound of the invention for use in methods of imaging one or more cells, organs or tissues comprising exposing cells to or administering to a subject an effective amount of a compound with an isotopic label suitable for imaging. In some embodiments, the one or more organs or tissues include prostate tissue, kidney tissue, brain tissue, vascular tissue or tumour tissue. The cells, organs or tissues may be imaged while within an organism, either by whole body imaging or intraoperative imaging, or may be excised from the organism for imaging.

In another embodiment, the imaging method is suitable for imaging of cancer, tumour or neoplasm. As used herein, the term “cancer” refers to or describes the physiological condition in mammals that is typically characterized by unregulated cell proliferation. The terms “cancer” and “cancerous” as used herein are meant to encompass all stages of the disease. A “cancer” as used herein is any malignant neoplasm resulting from the undesired growth, the invasion, and under certain conditions metastasis of impaired cells in an organism. The cells giving rise to cancer are genetically impaired and have usually lost their ability to control cell division, cell migration behaviour, differentiation status and/or cell death machinery. Most cancers form a tumour but some hematopoietic cancers, such as leukaemia, do not. A cancer generally forms at a primary site, giving rise to a primary cancer. Cancer that spreads locally, or to distant parts of the body is called a metastasis.

Thus, a “cancer” as used herein may include both benign and malignant cancers. A “cancer” as used herein may also include both primary and metastatic cancers. Examples of cancer include but are not limited to, carcinoma, lymphoma, blastoma, sarcoma, and leukaemia or lymphoid malignancies. More specifically, a cancer according to the present invention is selected from the group comprising squamous cell cancer (e.g., epithelial squamous cell cancer), lung cancer including small-cell lung cancer, non-small cell lung cancer, adenocarcinoma of the lung and squamous carcinoma of the lung, oropharyngeal cancer, nasopharyngeal cancer, laryngeal cancer, cancer of the peritoneum, oesophageal cancer, hepatocellular cancer, gastric or stomach cancer including gastrointestinal cancer and gastrointestinal stromal cancer, pancreatic cancer, glioblastoma, brain cancer, nervous system cancer, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, gallbladder cancer, vulval cancer, testicular cancer, thyroid cancer, Kaposi sarcoma, hepatic carcinoma, anal carcinoma, penile carcinoma, non-melanoma skin cancer, melanoma, skin melanoma, superficial spreading melanoma, lentigo maligna melanoma, acral lentiginous melanomas, nodular melanomas, multiple myeloma and B-cell lymphoma (including Hodgkin lymphoma; non-Hodgkin lymphoma, such as e.g., low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukaemia (CLL); acute lymphoblastic leukaemia (ALL); hairy cell leukaemia; chronic myeloblastic leukaemia (CML); Acute Myeloblastic Leukaemia (AML); and post-transplant lymphoproliferative disorder (PTLD), as well as abnormal vascular proliferation associated with phacomatoses, oedema (such as that associated with brain tumours), Meigs' syndrome, brain, as well as head and neck cancer, including lip a oral cavity cancer, and associated metastases.

In a preferred embodiment, said cancer is lung cancer, lip a oral cavity cancer, oropharyngeal cancer, nasopharyngeal cancer, laryngeal cancer, prostate cancer, oesophageal cancer, gallbladder cancer, liver cancer, hepatocellular cancer, gastric or stomach cancer including gastrointestinal cancer and gastrointestinal stromal cancer, pancreatic cancer, Hodgkin lymphoma, Non-Hodgkin lymphoma, leukaemia, multiple myeloma, Kaposi sarcoma, kidney cancer, bladder cancer, colon cancer, rectal cancer, colorectal cancer, hepatoma, hepatic carcinoma, anal carcinoma, thyroid cancer, non-melanoma skin cancer, skin melanoma, brain cancer, nervous system cancer, testicular cancer, cervical cancer, uterine cancer, endometrial cancer, ovarian cancer, or breast cancer.

In a more preferred embodiment, said cancer is oesophageal cancer, liver cancer, hepatocellular cancer, gastric or stomach cancer including gastrointestinal cancer and gastrointestinal stromal cancer, pancreatic cancer, Hodgkin lymphoma, colon cancer, rectal cancer, colorectal cancer, hepatoma, hepatic carcinoma, anal carcinoma, non-melanoma skin cancer, skin melanoma, cervical cancer, uterine cancer, endometrial cancer, ovarian cancer, or breast cancer.

The present inventors have found that the radio-labelled compounds described herein can be used to probe cancer in vitro and in vivo using autoradiographic techniques or molecular imaging modalities, such as PET or SPECT. The progastrin moiety binds specifically to the cancer cells, so that the signal emitted by the radioisotope indicates the localisation of the cancer cells.

According to another aspect, there is provided a method for imaging one or more cancer cells, organs or tissues in a subject in recognized need thereof, comprising:
a) administering a compound as described herein, or a pharmaceutically acceptable salt thereof, to said subject; and
b) detecting said compound by in vivo PET or SPECT imaging.

The compounds of the invention are also useful for diagnosing a cancer in a patient. According to this aspect, the invention provides a method of diagnosis of a cancer in a patient, said method comprising the steps of:
a) administering a compound as described herein, or a pharmaceutically acceptable salt thereof, to said subject;
b) detecting said compound by in vivo PET or SPECT imaging; and
c) diagnosing a cancer based on the detection of step b).

The present progastrin derivatives only bind cancer cells. Any signal detected in PET or SPECT imaging is thus an indication that cancer cells are present. Because of the sensitivity of the present radiolabelled compounds, it is possible to identify cancerous cells within the body of the patient, and thus diagnose a cancer. In addition, the cancer type can be readily deduced from the localisation of the primary cancer.

In another aspect, the present invention relates to a method of prognosis of a cancer in a patient, said method comprising the steps of:
a) administering a compound as described herein, or a pharmaceutically acceptable salt thereof, to said subject;
b) detecting said compound by in vivo PET or SPECT imaging; and
c) prognosing a cancer based on the detection of step c).

“Prognosis” as used herein means the likelihood of recovery from a disease or the prediction of the probable development or outcome of a disease. For example, the bigger the single detected in step b), the bigger the cancerous mass in the patient's body, the worse the prognosis.

In yet another aspect, the present invention provides a method of determining the localisation of a cancer in a subject in need thereof, comprising:
a) administering a compound as described herein, or a pharmaceutically acceptable salt thereof, to said subject; and
b) detecting said compound by in vivo PET or SPECT imaging.

It will be immediately clear to the skilled person that the invention also enables to identify the localisation of a cancer at the earliest stages. Notably, the present invention is particularly useful for identifying the site of a cancer which is too small to be detected otherwise. This is particularly advantageous when the sole indication that the patient has a cancer stems from the analysis of a biomarker. For example, an assay involving anti-progastrin antibodies and based on the detection of allows the identification of a risk of cancer even in the absence of any symptom (see e.g., WO 2017/114973).

According to a particular embodiment, the method of determining the localisation of a cancer in a subject in need thereof, comprises the steps of:
a) determining the level of progastrin in sample of said subject;
b) administering a compound as described herein, or a pharmaceutically acceptable salt thereof, to said subject; and
c) detecting said compound by in vivo PET or SPECT imaging.

The determination of the concentration of progastrin, in the present method, is performed by any technique known by one skilled in the art of biochemistry.

Preferably, determining the levels of progastrin in a sample includes contacting said sample with a progastrin-binding molecule and measuring the binding of said progastrin-binding molecule to progastrin.

When expression levels are measured at the protein level, it may be notably performed using specific progastrin-binding molecules, such as e.g., antibodies, in particular using well known technologies such as cell membrane staining using biotinylation or other equivalent techniques followed by immunoprecipitation with specific antibodies, western blot, ELISA or ELISPOT, enzyme-linked immunosorbant assays (ELISA), radioimmunoassays (RIA), immunohistochemistry (IHC), immunofluorescence (IF), antibodies microarrays, or tissue microarrays coupled to immunohistochemistry. Other suitable techniques include FRET or BRET, single cell microscopic or histochemistry methods using single or multiple excitation wavelength and applying any of the adapted optical methods, such as electrochemical methods (voltametry and amperometry techniques), atomic force microscopy, and radio frequency methods, e.g. multipolar resonance spectroscopy, confocal and non-confocal, detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry), cell ELISA, flow cytometry, radioisotopic, magnetic resonance imaging, analysis by polyacrylamide gel electrophoresis (SDS-PAGE); HPLC-Mass Spectroscopy; Liquid Chromatography/Mass Spectrometry/Mass Spectrometry (LC-MS/MS)). All these techniques are well known in the art and need not be further detailed here. These different techniques can be used to measure the progastrin levels.

Said method may in particular be chosen among: a method based on immuno-detection, a method based on western blot, a method based on mass spectrometry, a method based on chromatography, and a method based on flow cytometry. Although any suitable means for carrying out the assays are included within the invention, methods such as FACS, ELISA, RIA, western-blot and IHC are particularly useful for carrying out the method of the invention.

It was previously shown that the subject has a cancer if the level of progastrin is above 0 pM (see e.g., WO 2017/114973). According to a preferred embodiment, the method comprises the steps of:
a) measuring the level of progastrin in sample of said subject;
b) determining that the level of step a) is higher than 0 pM;
c) administering a compound as described herein, or a pharmaceutically acceptable salt thereof, to said subject; and
d) detecting said compound by in vivo PET or SPECT imaging.

By “progastrin-binding molecule”, it is herein referred to any molecule that binds progastrin, but does not bind gastrin-17 (G17), gastrin-34 (G34), glycine-extended gastrin-17 (G17-Gly), or glycine-extended gastrin-34 (G34-Gly). The progastrin-binding molecule of the present invention may be any progastrin-binding molecule, such as, for instance, an antibody molecule or a receptor molecule. Preferably, the progastrin-binding molecule is an anti-progastrin antibody or an antigen-binding fragment thereof. According to a particular embodiment of the method, the level of progastrin is determined by using one or more anti-progastrin antibodies. According to this embodiment, the level of progastrin is determined by contacting one or more anti-progastrin antibodies with the sample of said subject.

Said antibody may be a polyclonal or a monoclonal antibody. Preferably, the monoclonal anti-progastrin antibody of the present method is any of the monoclonal anti-hPG antibodies disclosed in WO 2017/114973.

A “biological sample” as used herein also includes a solid cancer sample of the patient to be tested, when the cancer is a solid cancer. Such solid cancer sample allows the skilled person to perform any type of measurement of the level of the biomarker of the invention. In some cases, the methods according to the invention may further comprise a preliminary step of taking a solid cancer sample from the patient. By a “solid cancer sample”, it is referred to a tumour tissue sample. Even in a cancerous patient, the tissue which is the site of the tumour still comprises non-tumour healthy tissue. The “cancer sample” should thus be limited to tumour tissue taken from the patient. Said “cancer sample” may be a biopsy sample or a sample taken from a surgical resection therapy.

A biological sample is typically obtained from a eukaryotic organism, most preferably a mammal, or a bird, reptile, or fish. Indeed, a “subject” which may be subjected to the method described herein may be any of mammalian animals including human, dog, cat, cattle, goat, pig, swine, sheep and monkey; or a bird; reptile; or fish. Preferably, a subject is a human being; a human subject may be known as a “patient”.

By “obtaining a biological sample,” it is herein meant to obtain a biological sample for use in methods described in this invention. Most often, this will be done by removing a sample of cells from an animal, but can also be accomplished by using previously isolated cells (e.g., isolated by another person, at another time, and/or for another purpose), or by performing the methods of the invention in vivo. Archival tissues, having treatment or outcome history, will be particularly useful.

This sample may be obtained and if necessary prepared according to methods known to a person skilled in the art. In particular, it is well known in the art that the sample should be taken from a fasting subject.

The determination of the concentration of progastrin relates to the determination of the quantity of progastrin in known volume of a sample. The concentration of progastrin may be expressed relatively to a reference sample, for example as a ratio or a percentage. The concentration may also be expressed as the intensity or localization of a signal, depending on the method used for the determination of said concentration. Preferably, the concentration of a compound in a sample is expressed after normalization of the total concentration of related compounds in said sample, for example the level or concentration of a protein is expressed after normalization of the total concentration of proteins in the sample.

Treatment prescribed to the cancer patient will be dependent upon the type of cancer. The present invention is particularly advantageous in this respect, as the type of cancer can be identified based on the localisation of said cancer in the patient. The appropriate therapy can be administered to the patient, thus improving his/her prognosis. The compounds described herein are especially useful, as they allow imaging and identification of a cancer at the earliest stages. Notably, when their use is coupled to the measurement of progastrin levels as described above, the present compounds allow the imaging and identification of a cancer even in the absence of any symptom. This is particularly useful for identifying the primary site of a cancer, since said cancer can be visualised before it has metastasised to distant parts in the body of the patient.

According to an aspect of the invention, a method of identifying the primary site of a cancer in a subject in need thereof is provided. This method comprises the steps of determining the localisation of the cancer by the methods described herein, and identifying the organ which is affected by the cancer. In an embodiment, the method further comprises an in vitro histological examination of a sample of said organ of said patient.

Another aspect of the present invention relates to a composition, notably a pharmaceutical composition, comprising a compound as described herein.

The compounds discussed herein can be formulated into various compositions, for use in diagnostic or imaging treatment methods. The compositions (e.g. pharmaceutical compositions) can be assembled as a kit.

Generally, a pharmaceutical composition comprises an effective amount (e.g., a pharmaceutically effective amount, or detectably effective amount) of a compound described above.

A composition of the disclosure can be formulated as a pharmaceutical composition, which comprises a compound of the invention and pharmaceutically acceptable carrier. By a “pharmaceutically acceptable carrier” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimise any degradation of the active ingredient and to minimise any adverse side effects in the subject, as would be well known to one of skill in the art. For a discussion of pharmaceutically acceptable carriers and other components of pharmaceutical compositions, see, e.g., Remington's Pharmaceutical Sciences, 18th ed., Mack Publishing Company, 1990. Some suitable pharmaceutical carriers will be evident to a skilled worker and include, e.g., water (including sterile and/or deionized water), suitable buffers (such as PBS), physiological saline, cell culture medium (such as DMEM), artificial cerebral spinal fluid, or the like.

A pharmaceutical composition or kit of the disclosure can contain other pharmaceuticals, in addition to the compound. The other agent(s) can be administered at any suitable time during the treatment of the patient, either concurrently or sequentially.

One skilled in the art will appreciate that the particular formulation will depend, in part, upon the particular agent that is employed, and the chosen route of administration. Accordingly, there is a wide variety of suitable formulations of compositions of the present disclosure.

One skilled in the art will appreciate that a suitable or appropriate formulation can be selected, adapted or developed based upon the particular application at hand. Dosages for compositions of the disclosure can be in unit dosage form. The term “unit dosage form” as used herein refers to physically discrete units suitable as unitary dosages for animal (e.g. human) subjects, each unit containing a predetermined quantity of an agent of the invention, alone or in combination with other therapeutic agents, calculated in an amount sufficient to produce the desired effect in association with a pharmaceutically acceptable diluent, carrier, or vehicle.

One skilled in the art can easily determine the appropriate dose, schedule, and method of administration for the exact formulation of the composition being used, in order to achieve the desired effective amount or effective concentration of the agent in the individual patient. The dose of a composition described herein, administered to an animal, particularly a human, in the context of the present invention should be sufficient to produce at least a detectable amount of a diagnostic response in the individual over a reasonable time frame. The dose used to achieve a desired effect will be determined by a variety of factors, including the potency of the particular agent being administered, the pharmacodynamics associated with the agent in the host, the severity of the disease state of infected individuals, other medications being administered to the subject, etc. The size of the dose also will be determined by the existence of any adverse side effects that may accompany the particular agent, or composition thereof, employed. It is generally desirable, whenever possible, to keep adverse side effects to a minimum. The dose of the biologically active material will vary; suitable amounts for each particular agent will be evident to a skilled worker.

The pharmaceutical or radiopharmaceutical composition may be administered parenterally, i.e., by injection, and is most preferably an aqueous solution. Such a composition may optionally contain further ingredients such as buffers; pharmaceutically acceptable solubilisers (e.g., cyclodextrins or surfactants such as Pluronic, Tween or phospholipids); pharmaceutically acceptable stabilisers or antioxidants (such as ascorbic acid, gentisic acid or para-aminobenzoic acid). Where the compound described herein is provided as a radiopharmaceutical composition, the method for preparation of said compound may further comprise the steps required to obtain a radiopharmaceutical composition, e.g., removal of organic solvent, addition of a biocompatible buffer and any optional further ingredients. For parenteral administration, steps to ensure that the radiopharmaceutical composition is sterile and apyrogenic also need to be taken. Such steps are well-known to those of skill in the art.

Other embodiments of the disclosure provide kits including a compound as disclosed herein, or a pharmaceutically acceptable salt thereof. In certain embodiments of the disclosure, the kit provides packaged pharmaceutical compositions having a pharmaceutically acceptable carrier and a compound as disclosed herein, or a pharmaceutically acceptable salt thereof. In some embodiments of the disclosure the packaged pharmaceutical composition will include the reaction precursors necessary to generate the compound as disclosed herein, or a pharmaceutically acceptable salt thereof, upon combination with a radionuclide. Other packaged pharmaceutical compositions provided by the present disclosure further comprise indicia comprising at least one of: instructions for preparing compounds as disclosed herein, or pharmaceutically acceptable salts thereof, from supplied precursors, instructions for using the composition to image cells or tissues, in particular instructions for using the composition to image cancer.

In certain embodiments of the disclosure, the present kit contains from about 1 mCi to about 30 mCi of the radionuclide-labelled imaging agent described above, in combination with a pharmaceutically acceptable carrier. The imaging agent and carrier may be provided in solution or in lyophilised form. When the imaging agent and carrier of the kit are in lyophilised form, the kit may optionally contain a sterile and physiologically acceptable reconstitution medium such as water, saline, buffered saline, and the like. The kit may provide a compound described herein in solution or in lyophilised form, and these components of the kit of the disclosure may optionally contain stabilisers such as NaCl, silicate, phosphate buffers, ascorbic acid, gentisic acid, and the like. Additional stabilisation of kit components may be provided in this embodiment, for example, by providing the reducing agent in an oxidation-resistant form. Determination and optimisation of such stabilisers and stabilisation methods are well within the level of skill in the art.

A “pharmaceutically acceptable carrier” refers to a biocompatible solution, having due regard to sterility, p[Eta], isotonicity, stability, and the like and can include any and all solvents, diluents (including sterile saline, Sodium Chloride Injection, Ringer's Injection, Dextrose Injection, Dextrose and Sodium Chloride Injection, Lactated Ringer's Injection and other aqueous buffer solutions), dispersion media, coatings, antibacterial and antifungal agents, isotonic agents, and the like. The pharmaceutically acceptable carrier may also contain stabilisers, preservatives, antioxidants, or other additives, which are well known to one of skill in the art, or other vehicle as known in the art.

The characteristics of the embodiments of the invention will become further apparent from the following detailed description of examples below.

### FIGURE LEGENDS

FIG. 1: 3D representation of quantified ROIs on PET/CT. Are represented the tumor (red), liver (blue), kidneys (green), heart (light blue), muscle (yellow) and brains (pink).

FIG. 2: Sagittal view of the dynamic PET/CT of the C1S3 mouse at different time point after injection 68Ga-NODAGA-Progastrin.

FIG. 3: Mean biodiversity of ⁶⁸Ga-NODAGA-Progastrin in each quantified organ during the 2 hours of PET. The values are expressed in % ID/g+standard deviation. (A) kinetics of all quantified regions, (B) kinetics restricted to muscle, brain and tumour

FIG. 4: Quantity in % ID/g 68Ga-NODAGA-peptide 1 measured in tumour and muscle (A) and Tumour to Muscle (B) ratio for each mouse after 2 hours of PET/CT acquisition.

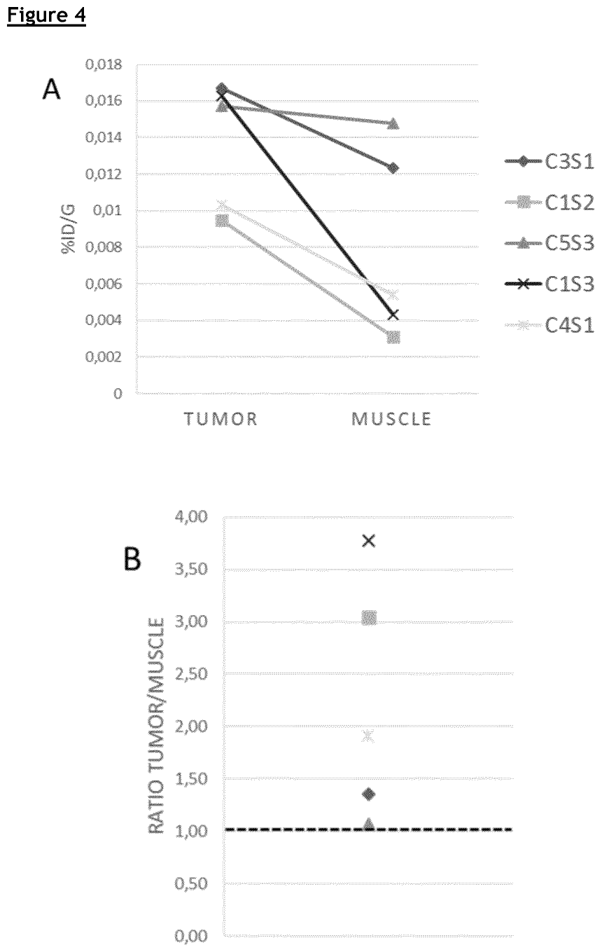

### EXAMPLES

Peptide Coupling

The chelator is prepared at a concentration of 10 mg/mL in a 0.2 M sodium bicarbonate solution at pH=9. Then, 10 equivalents of the chelator are added to an aliquot of progastrin. The conjugation reactions are carried out at 37° C. for 2 hours. The purification of the final product is carried out on AMICON filters. Through these filters, the excess unreacted chelator is removed. we obtain the conjugated peptide that we call NODAGA-Progastrin.

Animal Model

The colorectal cancer cell line T84 were cultured in T75 flask and passed 4 times after thawing to allow optimal growth rate to resume before xenograft in mice. The culture medium used was DMEM-F12 with Glutamax+10% fetal calf serum and 1% antibiotics (Streptomycin, Penicillin). For mouse xenograft, cell cultures are stopped at a confluence of 80% and the cells are taken up in a DMEM-F12 solution without Serum and Matrigel at a ratio of 1:1 to a concentration of 1·10⁹ cells/100 μl.

Mice are rapidly anesthetized with isoflurane and an injection of 100 μl of T84 (1·10⁹ cells/100 μl) is done in the subcutaneous area between the shoulder blades. The animals are put back in their cages as soon as their awakening and placed in a stable room until the tumour growth is sufficient to experimentation.

Radiolabelling and Imaging

100 μL of a 2 M ammonium acetate solution are added to an aliquot of NODAGA-Progastrin (10 μg solubilized in 50 μL of PBS). Then, 500 μL of gallium-68 eluate, [⁶⁸Ga]GaCl3, from an IRE Elit generator, are added to the previously prepared solution. The whole is incubated at room temperature for 10 minutes. The final pH is 4.8. The radiochemical purity is greater than 90% (n=3) and is determined by thin layer chromatography (mobile phase: 0.1 M sodium citrate at pH=5). 2 μL of 10 M sodium hydroxide are added to the final mixture to neutralize the pH. This prepared solution is used as is for biodistribution and PET/CT imaging studies.

The animals are put to sleep by gas anaesthesia (isoflurane at 3% for induction, and at 1·5-2% for mask maintenance). The caudal vein is catheterized (27G catheter). Mice receive a radiotracer injection in a bolus of 3.5±0.6 MBq for 2-hour dynamics (Table 2).

All PET/CT imaging is done with the nanoPET/CTO camera (Mediso, Hungary).

The animals are imaged 3 by 3. To obtain images of the biodistribution kinetics of the NODAGA-Progastrin, 2-hour dynamic PET images (400-600 keV energy window) combined with a scanner (35 kVp, 450 ms exposure time per projection) are performed over the entire mouse body (10 cm window). The PET acquisition starts 10 seconds before the radiotracer injection starts and allows the injection peak to be obtained. The PET images obtained are then reconstructed by applying an anatomical shift, attenuation correction and time division. The time division is as follows: 10″, 1″, 1′, 5′, 10′, 20′, 40′, 1h, 1h20′, 1h20′, 1h40′ and 2h.

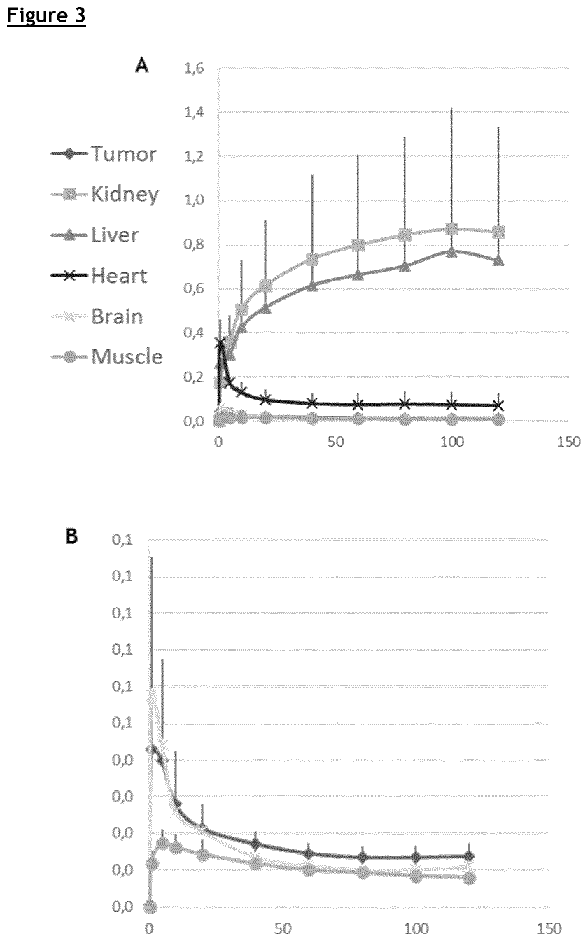

Post-analysis of the PET/CT 3D images was performed with VivoQuant 3.5 software (Invicro, USA). For dynamics, 6 regions of interest (ROIs) are detuned on the scanner, then transferred to the PET images for quantification. The quantified organs are the liver, kidneys, heart, brain, tumour and muscle (FIG. 3). The results of the quantifications are expressed either as a percentage of the dose injected per gram of tissue (% ID/g)* or as a Tumour/Muscle** ratio. * % ID/g=Activity calculated in ROI (MBq)/(Injected Activity (MBq)×Volume of tissue (ml))×100** The muscle is considered as a control region in the non-specific fixation of the radiotracer.

Results

In total, the biodistribution kinetics of NODAGA-Progastrin were monitored and quantified on a total of 5 mice that developed an ectopic tumour T84 between 100 and 600 mm3 (PET/CT acquisition in Table 2).

TABLE 2
Mouse-injected activity for dynamic 2-hour PET/CT acquisitions
and percentage purity of radiosynthesis

| Radiotracer | Acquisition | Mice | Injected activities MBq | % purity |
|---|---|---|---|---|
| NODAGA- | 1 | C3S | 3.78 | >95% |
| Progastrin | | C5S3 | 4.12 | |
| | 2 | C1S2 | 3.45 | 88% |
| | | C1S3 | 2.46 | |
| | | C4S1 | 3.31 | |

Tumour volumes of mice were measured on CT images (Table 3).

TABLE 3
Tumour volume at the time of imaging
calculated by clipping on the scanner

| Mice | C3S | C5S3 | C1S2 | C1S3 | C4S1 |
|---|---|---|---|---|---|
| Volume tumours in mm³ | 338 | 553 | 320 | 172 | 398 |

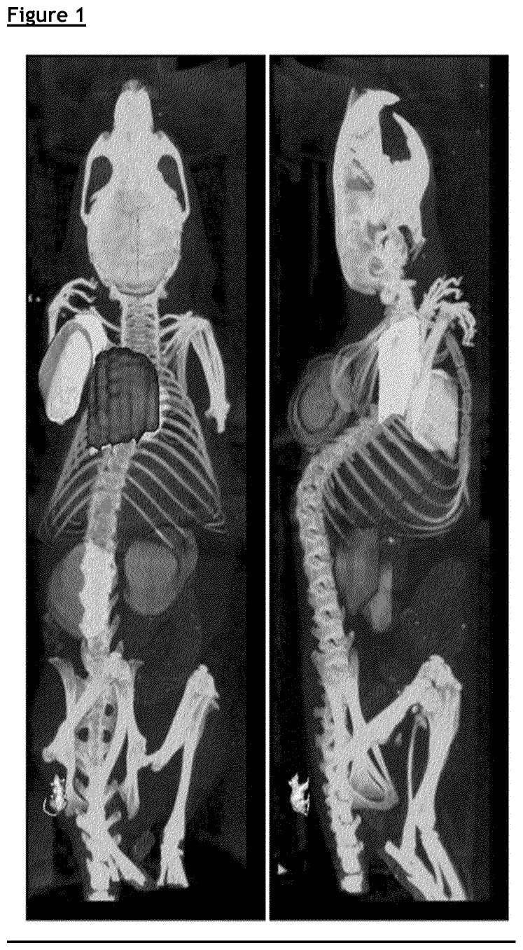

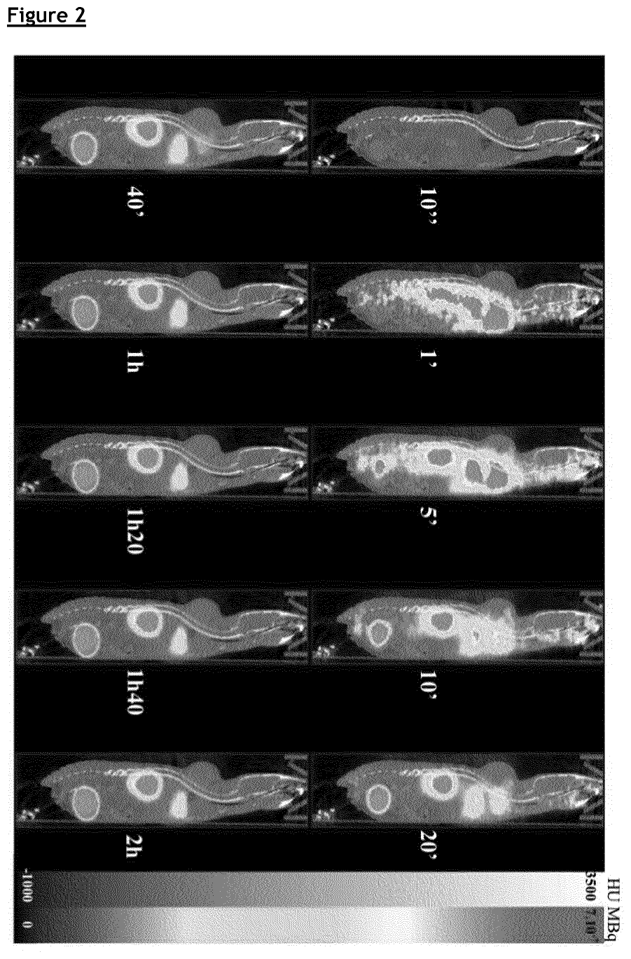

FIG. 1 illustrates the bio-allocation of this tracer during the 2 hours of PET imaging in a mouse. The average quantitation values in % ID/g of each interest region were calculated and are presented in FIG. 2.

As expected, we observed a high concentration in the elimination organs of the liver and kidneys and a much lower level of activity in the muscle or brain that does not specifically fix the tracer. More interestingly, the level of activity in the tumour is higher than in the muscle in the mice with a ratio Tumour/muscle ranking from 1 to 4 in the 5 mice (FIG. 3).

### CONCLUSION

We can conclude that there is an incorporation of radiolabelled Progastrin peptide into the tumour in this model.

1. A compound, or a pharmaceutically acceptable salt thereof, comprising:
a progastrin moiety, and
a chelating moiety.

2. The compound of claim 1, wherein the progastrin moiety is the peptide of sequence SEQ ID NO:1.

3. The compound of claim 1, wherein the chelating moiety is a bifunctional chelator.

4. The compound of claim 3, wherein the bifunctional chelator is selected from the group consisting of NODAGA, NOTA, DOTA, DOTA-NHS, p-SCN-Bn-NOTA, p-SCN-Bn-PCTA, p-SCN-Bn-oxo-DO3A, desferrioxamine-p-SCN, DTPA, and TETA.

5. The compound of claim 3, wherein the bifunctional chelator is NODAGA, NOTA, or DOTA.

6. The compound of claim 3, wherein the bifunctional chelator is NODAGA.

7. The compound of claim 1, further comprising a radioisotope selected from the group consisting of ⁶⁸Ga, ⁶⁴Cu, ⁸⁹Zr, ^{186/188}Re, ⁹⁰Y, ¹⁷⁷Lu, ¹⁵³Sm, ²¹³Bi, ²²⁵Ac, ¹¹¹In, ^{99m}Tc, ¹²³I, and ²²³Ra.

8. The compound of claim 7 any, wherein the radioisotope is ⁶⁸Ga or ⁶⁴Cu.

9. The compound of claim 7, wherein the radioisotope is ⁶⁸Ga.

10. A method of preparing the compound of claim 1, comprising the steps of:
a) conjugating an amine-reactive chelating moiety to the progastrin moiety; and
b) recovering the conjugate of progastrin and chelator.

11. The method of claim 10, wherein the amine-reactive chelating moiety is DOTA-NHS, NOTA-NHS, or NODAGA-NHS ester.

12. The method of claim 10, wherein the amine-reactive chelating moiety is NODAGA-NHS ester.

13. The method of claim 1, further comprising a step of:
c) incubating the conjugate of progastrin and chelator with the complementary radioisotope;
thus generating the compound of the invention.

14. A method of imaging one or more cancer cells, organs, or tissues in a subject in recognized need thereof, comprising:
a) administering a compound, or a pharmaceutically acceptable salt thereof, to said subject; and
b) detecting the compound by in vivo PET or SPECT imaging,
wherein the compound comprises:
a progastrin moiety, and
a chelating moiety.

15. A method of determining the localisation of a cancer in a subject in need thereof, comprising:
a) administering a compound, or a pharmaceutically acceptable salt thereof, to said subject; and
b) detecting the compound by in vivo PET or SPECT imaging:
wherein the compound comprises:
a progastrin moiety, and
a chelating moiety.

16. The method of claim 15, further comprising a prior step of determining the level of progastrin in sample of said subject.

17. The method of claim 16, wherein the level of progastrin is determined with anti-progastrin antibodies.

18. A pharmaceutical composition comprising a compound of claim 1 and a pharmaceutically acceptable carrier.

19. A kit comprising a compound of claim 1.
